(19) United States
(12) Patent Application Publication
(10) Pub. No.: US 2018/0042776 A1
(43) Pub. Date: Feb. 15, 2018

(54) TRANSPARENT BREATHABLE POLYURETHANE FILM FOR TATTOO AFTERCARE

(71) Applicant: TATUYOU, LLC, Hastings, MN (US)

(72) Inventors: Patrick F. McClernon, White Bear Lake, MN (US); Russell Blette, Hastings, MN (US); Donna Dearinger, San Jose, CA (US)

(21) Appl. No.: 15/792,211

(22) Filed: Oct. 24, 2017

Related U.S. Application Data
(62) Division of application No. 12/574,377, filed on Oct. 6, 2009, now abandoned.
(60) Provisional application No. 61/102,868, filed on Oct. 6, 2008.

(51) Int. Cl. A61F 13/02; A61F 15/00; A61F 13/00; A45D 44/12

(52) U.S. Cl. CPC A61F 13/0276; A61F 13/023; A61F 15/001; A61F 2013/00395; A61F 2013/0037; A61F 2013/00259; A61F 2013/00182; A45D 44/12; Y10T 428/249982; Y10T 156/10; Y10T 428/2848; Y10T 428/24942; Y10T 428/15; Y10T 428/13; A61F 2013/00868; A61F 2013/00846

(57) ABSTRACT

A tattoo aftercare assembly comprises an air-permeable transparent film such that the tattoo is visible through the film when applied thereto. The assembly further includes a carrier layer configured to be removable from the film. An adhesive is applied between the film and the carrier layer. A backing cover is positioned on an opposite side of the film than the carrier layer. The film and carrier layers are slightly narrower and shorter than the backing cover such that the backing cover is easily removable from the film. The film is configured for application over a newly-created tattoo for at least 12 hours, preferably up to 3 days.

### CROSS-REFERENCE TO RELATED APPLICATIONS

This Utility patent application claims priority to Provisional Application Ser. No. 61/102,868, filed on Oct. 6, 2008, which is incorporated herein by reference.

### BACKGROUND

The invention relates to a transparent, breathable, polyurethane film for covering a recently-created tattoo and method thereof.

The tattooing industry, and the techniques used therein, has been around for hundreds of years in one form or another. Tattooing is believed to have started in the Polynesian islands, and spread throughout the world by the sailors of Her Majesty's fleet of Great Britain.

The various techniques used for tattooing have not changed significantly over time, with the original patent for the present day tattoo machine being based on a Thomas Edison invention for a printing machine. The aftercare used in the tattooing industry has also not changed significantly, ranging from doing nothing, to applying homemade lotions, or balms, to the tattoo. Each tattoo artist has his or her own technique for aftercare.

For these and other reasons, there is a need for the present invention.

### BRIEF DESCRIPTION OF THE DRAWINGS

The accompanying drawings are included to provide a further understanding of the present invention and are incorporated in and constitute a part of this specification. The drawings illustrate the embodiments of the present invention and together with the description serve to explain the principles of the invention. Other embodiments of the present invention and many of the intended advantages of the present invention will be readily appreciated as they become better understood by reference to the following detailed description. The elements of the drawings are not necessarily to scale relative to each other. Like reference numerals designate corresponding similar parts.

FIG. 1A illustrates a tattoo cover assembly in accordance with one embodiment in a cross-sectional view.

FIG. 1B illustrates a tattoo cover assembly in accordance with one embodiment in a perspective view.

FIG. 2 illustrates a tattoo cover assembly and package in accordance with one embodiment in a cross-sectional view.

FIG. 3 illustrates a method of using a tattoo cover assembly in accordance with one embodiment.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments of the present invention can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

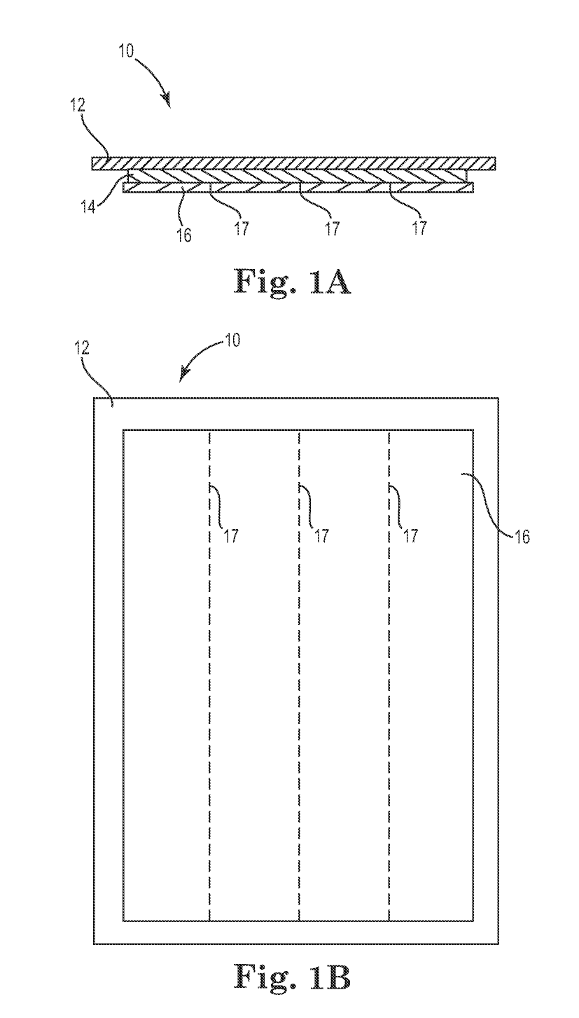

FIGS. 1A and 1B respectively illustrate cross-sectional and perspective views of a tattoo cover assembly 10 according to one embodiment. Tattoo cover assembly 10 includes backing cover 12, film 14, and carrier layer 16. An adhesive is applied to film 14 such that the adhesive is between film 14 and carrier layer 16.

In one embodiment, tattoo cover assembly 10 provides an effective way to cover a tattoo between sessions or after the tattooing process is complete. Once film 14 is applied over a tattoo, it helps prevent abrasion to the tattoo, it provides a sterile waterproof covering, and it allows for ready access to continue work on the new tattoo the next day. At the same time, in one embodiment, film 14 is translucent or transparent, and as such, film 14 still allows the person with the tattoo to both view and show the tattoo to others, without ever removing film 14.

In one embodiment, film 14 is translucent, such that light is allowed through it. In another embodiment, film 14 is transparent, such that the tattoo over which it is placed is clearly visible without having to remove film 14. Film 14 is flexible enough to allow for movement of the joints, and yet also prevents transfer of the tattoo inks and fluids to cloths or bedding while the tattoo is healing.

Film 14 can be made out of a variety of materials, for example, polyurethane, polyethylene, polypropylene, or a co-polymer. In one embodiment, film 14 is 3M™ Single coated polyurethane, Product Number 9832W. In another embodiment, film 14 is 3M™ Single coated polyurethane, Product Number 9833W. In yet another embodiment, film 14 is 3M™ Single coated incise medical tape, Product Number 9948.

In one embodiment, the adhesive backing carried on film 14 is an acrylate medical grade, but other types of medical grade adhesives can also be used in accordance with various embodiments. For example, certain synthetic rubber adhesives can be used. In one embodiment, film 14 has a thickness of 1.0 millimeters, and in another it has a thickness of 0.8 millimeters, and can also be less. In once embodiment, film 14 carries approximately 1.0 millimeters, or less, of adhesive.

Film 14 is applied by removing carrier layer 16, thereby exposing the adhesive carried on the surface of film 14. Film 14 is then smoothed onto the skin over the recently-applied tattoo. In one case, the carrier layer 16 has a scored line 17 down its center so that it is readily removed from film 14. In other embodiments, carrier layer 16 may have more scores 17 than just down its center (such as illustrated in FIG. 1A). Backing cover 12 can be removed before or after film 14 is smoothed into place over the tattoo. In one embodiment, film 14 and carrier layer 16 are slightly narrower and shorter than backing cover 12 (best illustrated in FIG. 1B), such that cover 12 is easily peeled away once film 14 is secured in place over the tattoo.

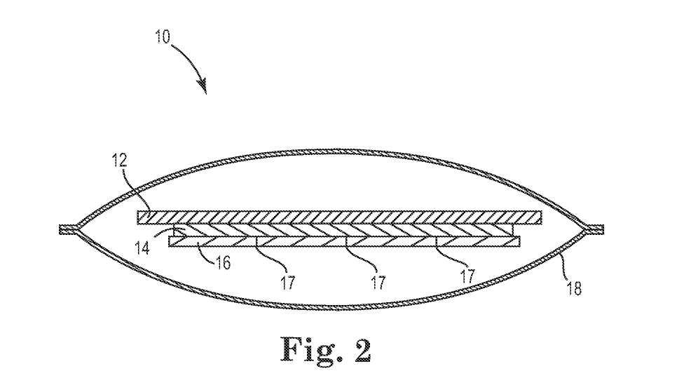

FIG. 2 illustrates illustrate a cross-sectional view of a tattoo cover assembly 10 within a package 18 according to one embodiment. Tattoo cover assembly 10 includes backing cover 12, film 14, and carrier layer 16, all of which are sealed within package 18. When package 18 is used to seal tattoo cover assembly 10, each of backing cover 12 and carrier layer 16 help prevent film 14 from adhering to the package itself, such that tattoo cover assembly 10 is easily removed from package 18 when it is opened.

In one embodiment, tattoo cover assembly 10 and package 18 are sterilized before sealing package 18 such that the film 14 will ultimately be sterile when applied to the tattoo. In one example, tattoo cover assembly 10 and package 18 are treated with Ethylene Oxide (EtO) sterilization. In that case, the EtO gas infiltrates package 18 as well as tattoo cover assembly 10 (including film 14) to kill micro organisms that are left behind during production or in the packaging processes. In another example, gamma irradiation sterilization is used to protect tattoo cover assembly 10 and package 18.

Tattoo artists recommend leaving a newly administered tattoo open and uncovered to heal. If a newly-created tattoo is briefly bandaged, they recommend removing it after an hour, and recommend never leaving it covered for more than two hours (see, Tattoo Aftercare, http://www.sharptattoos.com/aftercare.html). Most artists specifically counsel against covering a new tattoo and instruct that any covering, especially a plastic covering, be immediately removed (see, Tattoo Aftercare—How to Take Care of Your New Tattoo, About.Com, http://tattoo.about.com/cs/beginners/a/blaftercare.htm).

Directly contrary to these teachings, tattoo cover assembly 10 is configured to keep a newly administered tattoo covered for at least three, and as long as seven, days after its creation. Film 14 is configured to be permeable or “breathable”, in that it allows air to pass through it in order to keep the tattoo and adjacent skin oxygenated so that the skin can heal. In addition, because film 14 is transparent, the wearer will not be incentivized to remove the film in order to view the new tattoo, or in order to show it to others. As such, the wearer can leave film 14 in place for the duration of the healing process. This retains a barrier between the healing and open skin abrasions and outside contaminants. Also, it prevents transfer of bodily fluids, blood and oozing from the tattoo site, to the outside, such as onto clothes, sheets or other people.

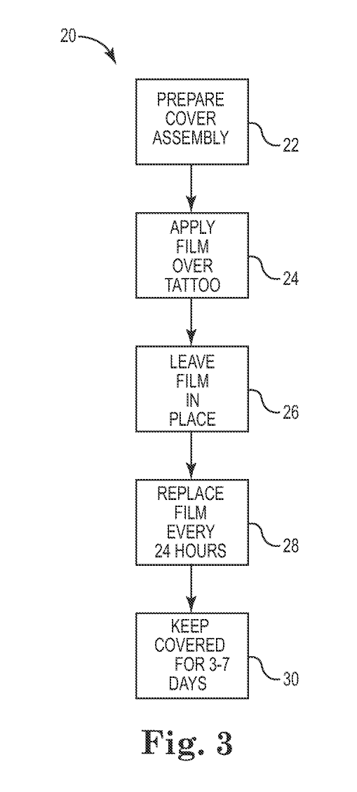

FIG. 3 illustrates a method of use 20 for tattoo cover assembly 10 according to one embodiment. In accordance with the method 20, once a tattoo is newly created, a user prepares tattoo cover assembly 10 for application at step 22. In one embodiment, this preparation includes removing the carrier layer 16 such that the adhesive carried on film 14 is exposed and ready for application over the tattoo. It may also first include unsealing package 18, which has been previously sterilized.

In step 24, film 14 is applied over a newly-created tattoo. In one embodiment, the adhesive backing carried on film 14 is an acrylate medical grade that will not stick to the tattoo itself due to the fact that a newly-administered tattoo oozes fluid, to which the adhesive will not adhere. Instead, the adhesive will adhere to the areas around the tattoo, thereby creating a seal over the tattoo to outside contaminants.

In some examples, film 14 in tattoo cover assembly 10 may not be large enough to cover the entire tattoo. For example, in one case, tattoo cover assembly 10 is 6 inches by 8 inches. Where a tattoo covers a significant portion of a person's back, for example, one film may not be enough to cover the entire work. In such a case, application of film 14 may include application of several films 14 that slightly overlap each other. The adhesive on film 14 sticks to film 14, and thus to other applied films as well, but not to the tattoo itself. As such, an entire area is readily covered with multiple films 14.

In step 26, film 14 is left in place over the tattoo for at least 12, and up to 24, hours. While the skin is still healing after a new tattoo is created, there is open tissue that is susceptible to infection. Film 14 provides an effective barrier to outside contaminants during this initial healing phase when the skin is especially vulnerable. Understandably, most people that have a new tattoo want to see it and to show it. Because film 14 is transparent, however, the wearer can still see and show the tattoo without removing the protective barrier provided by film 14 for the first 12 to 24 hours. Also, because film 14 is breathable and permeable to air, the healing process of the skin continues even while film 14 is in place covering the tattoo.

In step 28, film 14 is replaced at least every 24 hours. In order to ensure that the skin adjacent the tattoo continues to heal without infection, film 14 should not be left in place for more than 24 hours at a time. As such, sometime after 12 hours, and at least by 24 hours, film 14 is removed. At that time, the tattoo can be cleaned. Then, a new film 14 is placed over the tattoo and left for another 12 to 24 hours.

At step 30 this process is repeated for at least three and up to seven days. In this way, with method 20 a newly-created tattoo enjoys an effective barrier to outside contaminants for at least 3 days after the tattoo is made. At that same time, the tattoo continues to heal over that time with the benefit of film 14, which is breathable and permeable to air. The tattoo is also readily visible due to the transparency of film 14 for the entire duration. In some case, the tattoo site may be substantially healed by the time that film 14 is removed after seven days.

In an alternative embodiment, film 14 can be provided in roll form, such that various different lengths of film 14 can be pulled off the roll, and then the film can be cut to the desired length. In this way, a tattoo artist can have a roll of film 14 available while creating a tattoo. Once finished, the artist can pull off and cut the appropriate length of film 14 from the roll that will cover the newly-created tattoo. Even in roll form, film 14 can still have carrier layer 16 with adhesive applied between. Furthermore, roll of film 14 can be provided in a package 18, which can be sterilized as discussed above.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

1. A tattoo aftercare assembly comprising:
a film;
a carrier layer; and
an adhesive applied to the film between the film and the carrier layer wherein the film is configured for application over a newly-created tattoo for at least 12 hours.

2. The tattoo aftercare assembly of claim 1, wherein the film is configured to keep the newly-created tattoo covered for 3 days.

3. The tattoo aftercare assembly of claim 1, wherein the film is configured for replacement with a new film every 24 hours.

4. The tattoo aftercare assembly of claim 1, wherein the film comprises a transparent material such that the tattoo is visible through the film.

5. The tattoo aftercare assembly of claim 1, wherein the film comprises an air-permeable material such that air reaches the tattoo even when it is fully covered by the film.

6. The tattoo aftercare assembly of claim 1, wherein the film comprises one of a group of materials comprising, polyurethane, polyethylene, polypropylene, and co-polymer.

7. The tattoo aftercare assembly of claim 1 further comprising a package configured to completely encompass the film, the carrier layer and adhesive.

8. The tattoo aftercare assembly of claim 7 wherein the package, the film, the carrier layer and adhesive are treated with either Ethylene Oxide sterilization or gamma irradiation sterilization.

9. The tattoo aftercare assembly of claim 1 further comprising a backing cover on an opposite side of the film than the carrier layer.

10. The tattoo aftercare assembly of claim 9, wherein the film and carrier layers are slightly narrower and shorter than the backing cover such that the backing cover is easily removable from the film.

11. The tattoo aftercare assembly of claim 1, wherein carrier layer is scored in at least three locations for that the film is easily removable from the carrier layer.

12. A tattoo aftercare assembly comprising:
a film comprising an air-permeable transparent material such that the tattoo is visible through the film;
a carrier layer;
an adhesive applied to the film between the film and the carrier layer; and
a backing cover positioned on an opposite side of the film than the carrier layer, wherein the film is configured for application over a newly-created tattoo for at least 12 hours.

13. The tattoo aftercare assembly of claim 12, wherein the film and carrier layers are slightly narrower and shorter than the backing cover such that the backing cover is easily removable from the film.

14. The tattoo aftercare assembly of claim 12 further comprising a package configured to completely encompass the film, the carrier layer, the adhesive and the backing cover.

15. The tattoo aftercare assembly of claim 14 wherein the package, the film, the carrier layer and adhesive are treated with either Ethylene Oxide sterilization or gamma irradiation sterilization.

16. The tattoo aftercare assembly of claim 12, wherein the film is configured to keep the newly-created tattoo covered for 3 days.

17. A tattoo aftercare assembly comprising:
a film comprising an air-permeable transparent material such that the tattoo is visible through the film;
a carrier layer configured to be removable from the film;
an adhesive applied to the film between the film and the carrier layer; and
a backing cover positioned on an opposite side of the film than the carrier layer, wherein the film and carrier layers are slightly narrower and shorter than the backing cover such that the backing cover is easily removable from the film, wherein the film is configured for application over a newly-created tattoo for at least 12 hours.

18. The tattoo aftercare assembly of claim 17, wherein carrier layer is scored in at least three locations for that the film is easily removable from the carrier layer.

19. The tattoo aftercare assembly of claim 17 further comprising a package configured to completely encompass the film, the carrier layer, the adhesive and the backing cover.

20. The tattoo aftercare assembly of claim 19 wherein the package, the film, the carrier layer and adhesive are treated with either Ethylene Oxide sterilization or gamma irradiation sterilization.
